(19) United States
(12) Patent Application Publication
(10) Pub. No.: US 2021/0335492 A1
(43) Pub. Date: Oct. 28, 2021

(54) AUTOMATED TECHNIQUES FOR TESTING PROSPECTIVE MEMORY

(71) Applicant: KONINKLIJKE PHILIPS N.V., EINDHOVEN (NL)

(72) Inventors: Laura Klaming, Eindhoven (NL); Ron Dotsch, Haarlem (NL)

(21) Appl. No.: 17/284,176

(22) PCT Filed: Nov. 1, 2019
(86) PCT No.: PCT/EP2019/079946

§ 371 Date: Apr. 9, 2021

Related U.S. Application Data
(60) Provisional application No. 62/757,827, filed on Nov. 9, 2018.

(51) Int. Cl. G16H 50/20; G16H 10/20; G16H 40/63; A61B 5/16

(52) U.S. Cl. CPC G16H 50/20; A61B 5/162; G16H 40/63; G16H 10/20

(57) ABSTRACT

Embodiments described herein relate to automated digital prospective memory tests. In various embodiments, working memory instructions may be provided (402) that prompt a subject to perform working memory task(s). Additional instruction(s) may also be provided (404) prompting the subject to perform prospective memory task(s). Each additional instruction may prompt the subject to perform a respective prospective memory task after a predetermined time interval. During the predetermined time interval, the subject's performance of the working memory task(s) may be monitored (406) to determine a measure of engagement. An actual time interval between provision of a given additional instruction and performance of the prospective memory task prompted by the given additional instruction may be determined (408). If the measure of engagement satisfies a criterion, a certified prospective memory measure of the subject, calculated based at least in part on the actual time interval, may be provided (412).

### TECHNICAL FIELD

Various embodiments described herein are directed generally to health care. More particularly, but not exclusively, various methods and apparatus disclosed herein relate to automated techniques for digital testing prospective memory in subjects.

### BACKGROUND

Prospective memory is part of episodic memory and refers to our memory for future events. Prospective memory problems are common in elderly people and people with cognitive impairment, e.g. due to neurodegenerative disease or acquired brain damage. Currently, digital tests for prospective memory are lacking. Paper-pencil tests of prospective memory are difficult to administer, impractical, require an expert examiner, and are therefore not ideal for the purpose of assessing or monitoring an examinee's prospective memory.

### SUMMARY

Given the issues set forth previously, it would be beneficial to have a simple, easy-to-administer, digital test of prospective memory. Accordingly, the present disclosure is directed to methods and apparatus for automated testing of prospective memory in subjects. Subjects may interact with various client devices (e.g., tablet computers, smart phones, laptop computers, standalone interactive speakers with or without displays, wearable technology such as smart watches or smart glasses, etc.) to participate in prospective memory tests. These tests may prompt the subjects to interact with various types of input components (e.g., touchscreens, keyboards, mice, microphones, vision sensors, etc.) to perform a variety of different types of tasks. The subjects' interaction with these input components may be monitored, e.g., on a time scale, to determine prospective memory measures of the subjects. These prospective memory measures may take various forms (e.g., between zero and one on a scale, between zero and one hundred, letter grades, points, etc.) and may be indicative of prospective memory of the subjects (e.g., healthy, unhealthy, declining, improving, etc.).

For example, in various embodiments, a subject (who when being tested using techniques described herein may also be referred to as a “patient”, “user” or “examinee”) may be instructed, e.g., via output provided by one or more client devices operated or controlled by the subject, to perform one or more “working memory” or “filler” tasks. These tasks may be selected to occupy or strongly engage the subject's working memory. Meanwhile, the subject may also be asked to perform one or more prospective memory tasks that are selected to gauge the subject's prospective memory. In particular, the subject may be asked to perform a prospective memory task at some future point in time, e.g., in x minutes, y minutes after some audio, visual, or haptic cue, etc. (unless otherwise indicated, all numeric indicators described herein are positive integers). Performing the prospective memory task may require a subject to interact with one or more input components of the same client device that provided the instructions or a different client device. A difference or delta (Δ) between an actual time at which the subject performs the prospective memory test and the instructed time to perform the test may be determined and used to determine a measure of the subject's prospective memory. In some cases, the greater the delta, the worse the subject's prospective memory. Additionally or alternatively, in some embodiments, whether the subject performs the prospective memory task correctly before or after the instructed point in time may also be considered.

Generally, in one aspect, a method for conducting a prospective memory test on a subject may include: providing, via one or more output devices, working memory instructions that prompt the subject to perform one or more working memory tasks, wherein the one or more working memory tasks are selected to occupy the working memory of the subject; providing, via one or more of the output devices, one or more additional instructions prompting the subject to perform one or more prospective memory tasks, wherein each respective additional instruction of the one or more additional instructions prompts the subject to perform a respective one of the prospective memory tasks a predetermined time interval after provision of the respective additional instruction; during the predetermined time interval, monitoring performance by the subject of the one or more working memory tasks, wherein the monitoring includes determining a measure of engagement between the subject and one or more input devices operated by the subject to perform the one or more working memory tasks; determining an actual time interval between provision of a given additional instruction of the one or more additional instructions and performance of the prospective memory task prompted by the given additional instruction; determining whether the measure of engagement satisfies a criterion; conditionally providing a certified prospective memory measure of the subject based on determining that the measure of engagement satisfies the criterion, wherein the certified prospective memory measure is calculated based at least in part on the actual time interval; and conditionally providing a notification that the subject was not adequately engaged with the one or more working memory tasks based on determining that the measure of engagement satisfies the criterion.

In various embodiments, the method may further include rendering, on a display, a graphical user interface, wherein the graphical user interface is operable by the subject to participate in the prospective memory test. In various embodiments, providing the working memory instructions may include providing, as part of the graphical user interface, one or more working interactive elements that are operable by the subject to perform the one or more working memory tasks. In various embodiments, providing the one or more additional instructions may include providing, as part of the graphical user interface, one or more prospective memory interactive elements that are operable by the subject to perform the one or more prospective memory tasks.

In various embodiments, the actual time interval may occur between provision of the one or more prospective memory interactive elements and operation of the one or more prospective memory interactive elements by the subject. In various embodiments, the method may further include rendering, as part of the graphical user interface, a clock.

In various embodiments, the respective additional instruction may be provided immediately prior to the one or more working memory instructions. In various embodiments, the respective additional instruction may be provided after the one or more working memory instructions.

In addition, some implementations include one or more processors of one or more computing devices, where the one or more processors are operable to execute instructions stored in associated memory, and where the instructions are configured to cause performance of any of the aforementioned methods. Some implementations also include one or more transitory or non-transitory computer readable storage media storing computer instructions executable by one or more processors to perform any of the aforementioned methods.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating various principles of the embodiments described herein.

FIG. 1 illustrates an example environment in which selected aspects of the present disclosure may be implemented, in accordance with various embodiments.

FIG. 2 depicts on example scenario in which techniques described herein are performed using visual output and touchscreen input.

FIGS. 3A and 3B depict example scenarios in which techniques described herein are performed using audio output and audio and/or visual input.

FIG. 4 depicts an example method of performing selected aspects of the present disclosure, in accordance with various embodiments.

FIG. 5 depicts an example computer architecture on which selected aspects of the present disclosure may be implemented.

### DETAILED DESCRIPTION

Prospective memory is part of episodic memory and refers to our memory for future events. Prospective memory problems are common in elderly people and people with cognitive impairment, e.g. due to neurodegenerative disease or acquired brain damage. Currently, digital tests for prospective memory are lacking. Paper-pencil tests of prospective memory are difficult to administer, impractical, require an expert examiner, and are therefore not ideal for the purpose of assessing or monitoring an examinee's prospective memory.

Applicants have recognized and appreciated that it would be beneficial to have a simple, easy-to-administer test of prospective memory in subjects. Accordingly, the present disclosure is directed to methods and apparatus for automated testing of prospective memory in subjects. Subjects may interact with various client devices to participate in prospective memory tests. These tests may prompt the subjects to interact with various types of input components (e.g., touchscreens, keyboards, mice, microphones, vision sensors, etc.) to perform a variety of different types of tasks. The subjects' interaction with these input components may be monitored, e.g., on a time scale, to determine prospective memory measures of the subjects. These prospective memory measures may be indicative of the subject's, prospective memory (e.g., healthy, unhealthy, declining, improving, etc.).

For example, in various embodiments, a subject (who when being tested using techniques described herein may also be referred to as a “patient”, “user” or “examinee”) may be instructed, e.g., via output provided by one or more client devices operated or controlled by the subject, to perform one or more “working memory” or “filler” tasks. These tasks may be selected to or strongly engage or occupy the subject's working memory. Meanwhile, the subject may also be prompted to perform one or more prospective memory tasks that are selected to obtain a measure of the subject's prospective memory. In particular, the subject may be asked to perform a prospective memory task at some future point in time, e.g., in x minutes, y minutes after some audio, visual, or haptic cue, etc. Performing the prospective memory task may require the subject to interact with one or more input components of the same client device that provided the instructions or a different client device. A difference or delta (Δ) between an actual time at which the subject performs the prospective memory test and the instructed time to perform the test may be determined and used to determine a measure of the subject's prospective memory. In some cases, the greater the delta, the worse the condition of the subject's prospective memory. Additionally or alternatively, in some embodiments, whether the subject performs the prospective memory task correctly before or after the instructed point in time may also be considered.

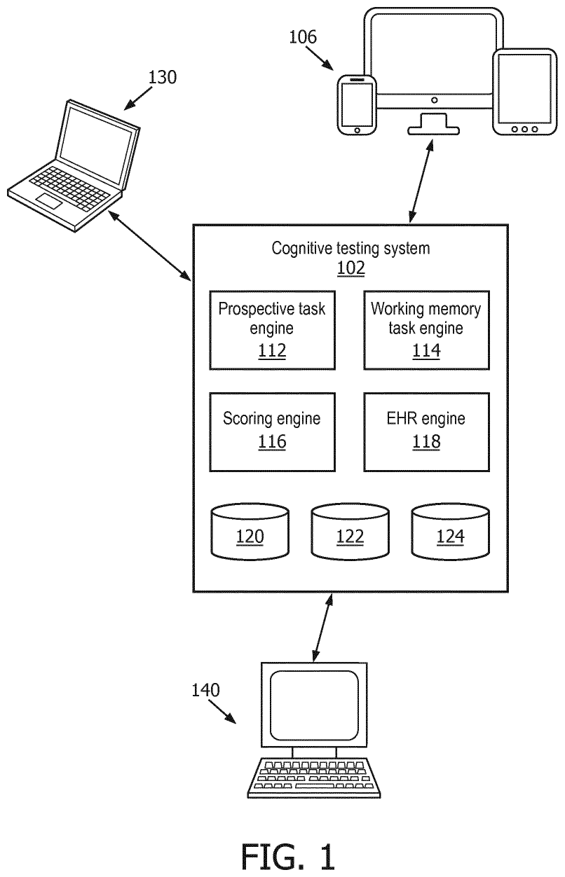

Referring to FIG. 1, an example environment in which selected aspects of the present disclosure may be implemented is depicted schematically. A cognitive testing system 102 may be implemented on one or more computing systems (e.g., blade servers) communicatively coupled via one or more computing networks (not depicted). Cognitive testing system 102 may be communicatively coupled over one or more computing networks (not depicted) with various computing devices operated by various parties. For example, in FIG. 1, cognitive testing system 102 is communicatively coupled with one or more client devices 106 operated by one or more subjects (not depicted) to be tested. Cognitive testing system 102 is also communicatively coupled with one or more medical personnel computing devices 130 that may be operated, for instance, by medical personnel charged with the care of the subjects that operate client devices 106. Other computing devices 140 may be communicatively coupled with cognitive testing system 102 as well, such as computing devices operated by researchers, insurance companies, subjects' family members and/or caregivers, and so forth.

The computing devices depicted in FIG. 1 (e.g., 106, 130, 140) may include, for example, one or more of: a desktop computing device, a laptop computing device, a tablet computing device, a mobile phone computing device, a computing device of a vehicle of the user (e.g., an in-vehicle communications system, an in-vehicle entertainment system, an in-vehicle navigation system), a standalone interactive speaker (which in some cases may include a vision sensor), a smart appliance such as a smart television (or a standard television equipped with a networked dongle with automated assistant capabilities), and/or a wearable apparatus of the user that includes a computing device (e.g., a watch of the user having a computing device, glasses of the user having a computing device, a virtual or augmented reality computing device). Additional and/or alternative client computing devices may be provided.

Client devices 106 may include various types of input and output components. The output components may be used to provide instructions to subjects to perform various types of tasks, such as working memory tasks, prospective memory tasks, and so forth. In some embodiments, output components of client devices 106 may also be used to provide feedback to subjects, such as whether the subjects' measures of prospective memory (or other cognitive measures) are improving, deteriorating, steady, and so forth. Output components of client devices 106 may include, but are not limited to, displays (touchscreen or otherwise), speakers, haptic feedback devices (e.g., to cause vibration), olfactory emitters, lights such as light-emitting diodes, printers, wireless transmitters (which may be part of wireless transceivers), network cards, and so forth.

Input components of client devices 106 may include, but are not limited to, keyboards, mice, touchscreens, microphones, vision sensors (e.g., RGB cameras, infrared sensors, etc.), network cards, wireless receivers (which may be part of wireless transceivers), various types of physiological sensors, retina scans, fingerprint scans, and so forth. Subjects may interact with input components, e.g., in response to instructions received via output components, in order to perform various types of tasks, such as the aforementioned working and prospective memory tasks. In some embodiments, the subjects' engagement with input components may be monitored, e.g., by one or more processors local to client device 106 and/or one or more components of cognitive testing system 102, e.g., to determine states of the subjects' prospective memories.

In some implementations, client devices 106 operated by a particular subject may form part of a coordinated “ecosystem” of client devices 106. This ecosystem may include any client device that the subject has used to “sign into” an online profile. In some embodiments, multiple client devices of the ecosystem may be employed, e.g., simultaneously or at different times, in order to perform cognitive testing. Examples of such deployment will be described below.

Medical personnel such as psychologists, doctors, nurses, therapists, psychiatrists, counselors, etc., may operate computing devices 130 to receive and/or analyze results of prospective memory testing performed by subjects using client devices 106. Additionally or alternatively, other computing devices 140 may be operated by interested parties, such as researchers, subject family members, caregivers, insurance companies, etc., to receive and/or analyze results of prospective memory testing performed by subjects using client devices 106.

Cognitive testing system 102 may include a variety of different components that may operate across one or more computing systems, such as one or more blade servers. In some implementations, one or more components of cognitive testing system 102 may form what is often referred to as a “cloud” infrastructure. In those situations, those components may be referred to as “cloud-based” because a party interacting with them (e.g., a subject, medical personnel, etc.) need not know the particulars about how the servers are configured.

A prospective task engine 112 may be configured to select and cause to be provided, at one or more client devices 106 operated by one or more subjects, instructions to perform various prospective memory tasks. The nature of the prospective memory tasks is not as important as the fact that the subject is instructed to complete the prospective memory tasks at some point in the future. For example, a prospective memory task instruction (which may be displayed or output audibly) may prompt a subject to perform some arbitrary action, such as interacting somehow with one or more graphical elements rendered on a graphical user interface (“GUI”), clapping, speaking a word or phrase, performing some gesture, and so forth. However, the time at which the subject performs the prospective memory tasks may be monitored to determine how precisely, temporally-speaking, the subject was able to follow the prospective memory instruction. For example, the instruction may prompt the subject to perform some action in x seconds, y minutes, etc. Additionally or alternatively, the instruction may prompt the subject to perform a prospective memory task at some future point in time after receiving some cue. For example, an instruction may be, “Touch the yellow star two minutes after it appears on your screen.”

In some implementations, various prospective tasks may be stored in a prospective task library 120 (or “database”) such that they are accessible to prospective task engine 112. In some such embodiments, prospective task engine 112 may select prospective tasks randomly and/or based on various heuristics or statistical models. For example, subjects diagnosed with one condition may be better tested using one type of prospective memory tasks, whereas other subjects may be better tested using another type of prospective memory tasks. Also, for instance, prospective task engine 112 may select appropriate tasks for visually-impaired and/or hearing-impaired subjects, e.g., so that those subjects' prospective memories are tested, not their abilities to see and/or hear.

Working memory task engine 114 may operate similarly to prospective task engine 112, except that instead of providing prospective memory tasks, working memory task engine 114 may provide what are referred to herein as “working memory,” “working,” or “filler” tasks. Working memory tasks may be selected and implemented in order to occupy the working memory of the subject. Without sufficient engagement with one or more working memory tasks, any prospective memory test performed by the subject may not be a reliable indicator of the subject's prospective memory condition. By requiring the subject to engage in working memory task(s), performance of prospective memory tasks by the subject can be measured.

Working memory tasks can take a variety of different forms. For example, a working memory task in some embodiments may continue until all desired prospective memory tasks have been administered. Examples of working memory tasks include, but are not limited to, playing a game, consuming media, speaking well-known phrases, reading text, and so forth. In some implementations, working memory task engine 114 may select working memory tasks from a working memory task library 122 (or “database”). As with prospective memory tasks, working memory tasks may be selected from working memory task library 122 at random, based on various heuristics, using various statistical models, etc.

Scoring engine 116 may be configured to analyze various signals to determine a measure of prospective memory of a subject. These signals may come in various forms. As noted previously, if the subject is not sufficiently engaged in the working memory task, he/she may use his/her working memory to perform the prospective memory tasks instead of using his/her prospective memory to perform the prospective memory tasks. Thus, in some embodiments, scoring engine 116 may monitor performance by the subject of one or more working memory tasks, e.g., by monitoring interaction between the subject and one or more input components of client device(s) 106. For example, the monitoring may include determining a measure of engagement between the subject and one or more input devices operated by the subject to perform the one or more working memory tasks. In various embodiments, scoring engine 116 may determine whether the measure of engagement determined for a subject satisfies one or more criterion. Various criteria may be analyzed. For example, in some embodiments, the criteria may be whether the subject provided input at some predetermined frequency, whether the subject advanced through working memory task(s) at a sufficient speed, whether the subject worked steadily to complete the working memory task(s), etc.

Assuming the subject is sufficiently engaged with the working memory task(s), in various embodiments, scoring engine 116 may be configured to determine (e.g., calculate) a measure of prospective memory of the subject based on the subject's response time for completing prospective memory task(s) provided by prospective task engine 112. For example, in some implementations, an algorithm such as that described below may be employed by scoring engine 116 (or another component of cognitive testing system 102).

In some embodiments, a subject (or “participant”) may receive n prospective memory tasks. Each prospective memory task i may be defined by: a timepoint tᵢ since start of the prospective memory task that defines the moment the prospective memory task should be performed; an optional cue cᵢ (e.g., containing text) that will be presented at timepoint t^{cue}ᵢ; and an action aᵢ that should be performed at tᵢ in order to complete the prospective memory task. In some embodiments, the algorithm may further include two general action interval parameters, ∂_{before} and ∂_{after}, which specify how much actual time before tᵢ and after tᵢ an action is allowed to take place in order to be counted as correct timing of the prospective memory task.

Each action performed by a subject, which may range from 1 to k total number of performed actions during one or more working memory task(s) may be logged as performed action a^{performed}ⱼ, at time t^{performed}ⱼ, where j denotes a the j^{th} performed action. The scoring algorithm may then proceed to compute the score of an individual, e.g., as follows (in pseudocode):
score=0
foreach (task i in all prospective memory tasks):
   foreach (performed action j in all performed actions):
      if aᵢ==a^{performed}ⱼ:
      score=score+1
         if t^{performed}ⱼ>tᵢ−∂_{before} and t^{performed}ⱼ<tᵢ+∂_{after}:
            score=score+1
         break out of inner loop
            In this algorithm, the outermost foreach statement loops through each of the n prospective memory tasks. In the next foreach statement, all j actions performed by the subject, whether as part of performing a prospective memory task or as part of performing a working memory task, may be considered. If the current action aᵢ is the same as the a^{performed}ⱼ, then the subject's prospective memory score (or “prospective memory measure”) may be incremented by one. Otherwise, the next action aⱼ₊₁ performed by the subject may be considered.

Assuming the subject's prospective memory score is incremented, it may then be determined whether the subject performed the prospective memory task within a predetermined time interval of the scheduled timepoint tᵢ (if t^{performed}ⱼ>tᵢ−∂_{before} and t^{performed}ⱼ<tᵢ+∂_{after}). If the answer is yes, then the subject's prospective memory score may be incremented again. Otherwise, the algorithm may break out of the inner loop and move to the next prospective memory task i+1. Of course, this is just one example of an algorithm that may be implemented by scoring engine 116 to determine a subject's prospective memory measure, and is not meant to be limiting.

In some implementations, a subject's performance may be compared to previous performance(s) by the same subject on the same prospective memory test and/or to normative data. This algorithm enables multiple alternate versions for repeated testing of a subject. Difficulty of the test for a given subject can be varied if necessary/desired, e.g., by increasing the number of prospective and/or working memory tasks administered or increasing the difficulty of the working memory task(s).

Electronic health records (“EHR”) engine 118 may have access to an EHR database 124 that stores information about subjects, such as their electronic health records, results of tests (including prospective memory tests described herein), demographics, health histories, etc. This data may be used for a variety of purposes related to prospective memory testing. For example, as noted above, one or both of engines 112 and 114 may select tasks based at least in part on a subject's medical data. If the subject is visually-impaired and/or hearing-impaired, then prospective memory and/or working memory tasks may be selected that minimize or eliminate the influence of these impairments. If the subject is already known (based on data in database 124) to be experiencing cognitive decline, then tasks and/or difficulties associated with tasks may be selected, e.g., by engines 112 and/or 114, based on signals provided by EHR engine 118. Additionally or alternatively, in some embodiments, results of prospective memory tests administered using techniques described herein may be stored by EHR engine 118 in EHR database 124.

While particular components are depicted in FIG. 1, this is not meant to be limiting. One or more functions described above as being performed by one component could be implemented by another component. Moreover, functionalities described in association with various components above may be combined into lesser numbers of components, such as a single component. In addition, in various implementations, various components of cognitive testing system 102 may be implemented using any combination of hardware or software. Moreover, one or more functionalities described above as being performed by component(s) of cognitive testing system 102 may be performed, in whole or in part, elsewhere from cognitive testing system 102, such as on one or more client devices 106.

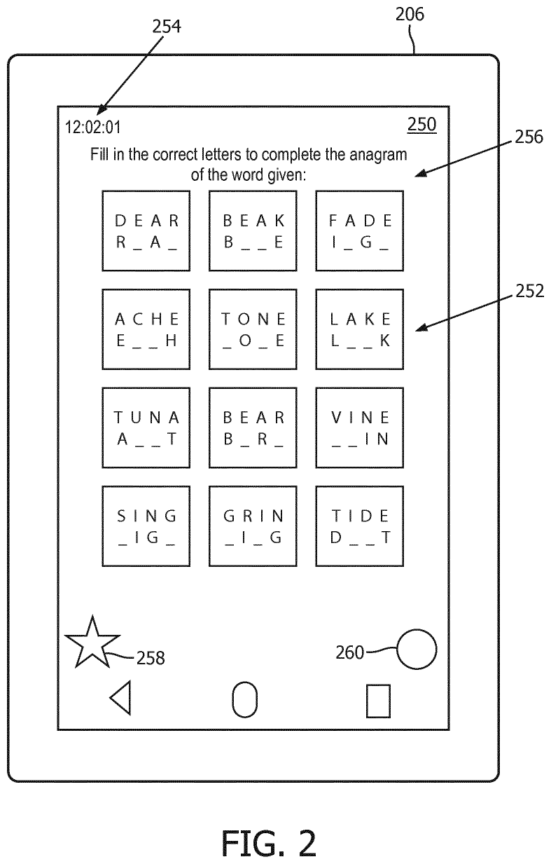

Referring now to FIG. 2, one non-limiting example of how techniques described herein may be employed to administer a prospective memory test is illustrated. A client device 206 configured with selected aspects of the present disclosure takes the form of a tablet computer or smart phone equipped with a touchscreen 250. However, this is not meant to be limiting, and client device 206 may take other forms as well. In FIG. 2, client device 206 renders a GUI 252 that is operable by a subject to participate in a prospective memory test that incorporates selected aspects of the present disclosure. As part of GUI 252, in various implementations, a clock 254 or other time tracking graphical element may be rendered, e.g., so that the subject knows how much time has elapsed and hence is able to perform prospective memory tasks on schedule (at least to the best of their ability).

Also rendered as part of GUI 252 is a working memory task instruction 256 that instructs the subject to “Fill in the correct letters to complete the anagram of the word given.” While provided visually in FIG. 2, this is not required, and in other embodiments, such instructions may be provided using other output modalities, such as audibly. Below working memory task instruction 256 are a number of graphical elements that each includes a complete word and an incomplete word for which the subject is supposed to provide missing letters. This working memory task takes the form of a word game, but any type of working memory task may be used so long as it sufficiently occupies the working memory of the subject.

Also rendered as part of GUI 252 are two graphical elements, 258 and 260, that are operable by the subject, e.g., by tapping or swiping. While the subject performs the working memory task of filling in the anagrams, the subject may be provided with prospective memory instructions that prompt the subject to interact with these elements 258, 260 at selected future points in time. For example, client device 206 may audibly provide instructions for the subject to tap the star 258 in three minutes. As another example, client device 206 may render on GUI 252 a pop-up window or other graphical element (not depicted in FIG. 2) that prompts the subject to tap on the circle 260 one minute after it flashes some color.

Thus, as the subject performs the working memory task of filling in the anagrams, he or she also must use prospective memory to remember which prospective memory tasks to perform at what time point. As described above, the delta between the scheduled prospective memory task completion times and the actual (observed) prospective memory task completion times may be used to determine a measure of the subject's prospective memory. Meanwhile, the subject must remain engaged with the working memory task, e.g., by interacting with at least one of the displayed graphical elements every so often. If the subject's interactions with touchscreen 250 fall below some threshold, the outcomes of the subject's performance of the prospective memory tasks may be discarded. Additionally or alternatively, the outcomes of the subject's performance of the prospective memory tasks may be negatively influenced by insufficient engagement with the working memory task(s) (e.g., if the subject stops performing the working memory task(s) but performs the prospectively memory task(s) in a timely manner, that may be interpreted as having a negative or at least neutral impact on the subject's prospective memory measure or score).

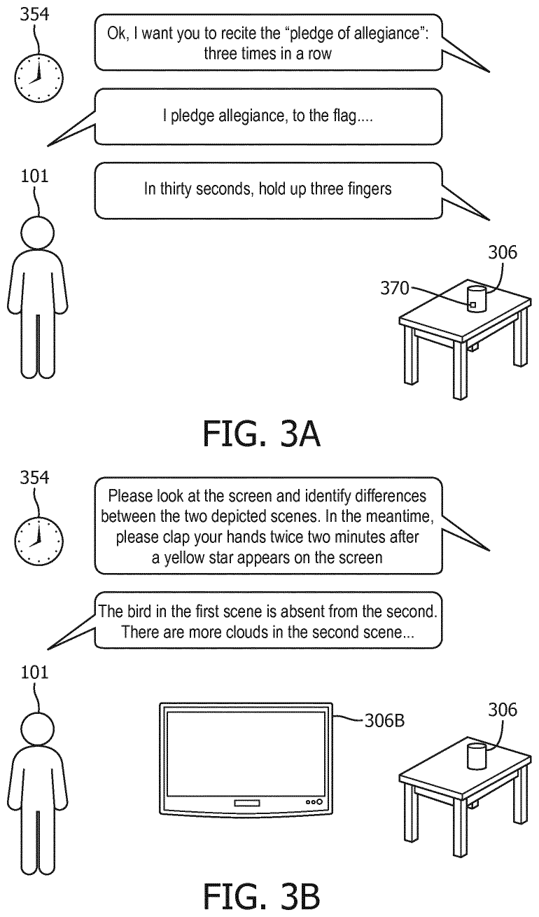

FIGS. 3A and 3B depict two more examples of scenarios in which techniques of the present disclosure are deployed. Unlike the example of FIG. 2, in FIGS. 3A and 3B, client device 306 takes the form of a standalone interactive speaker that is becoming increasingly commonplace in homes and businesses. Also unlike FIG. 2, a subject 101 participates in the prospective memory using audio and visual input devices, rather than a touchscreen. In FIG. 3A, client device 306 is equipped with a microphone (not depicted) and a vision sensor 370. In FIG. 3B, client device 306 includes a microphone (not depicted) but not a vision sensor. In both figures, a clock 354 is provided so that subject 101 is able to keep track of time for purposes of performing prospective memory tasks. Such a clock could alternatively be rendered on a client device display if available.

In FIG. 3A, working memory task instructions are provided to the subject 101, “OK, I want you to recite the pledge of allegiance three times in a row.” While subject 101 begins reciting, a prospective memory task instruction is provided, “in thirty seconds, hold up three fingers.” Client device 306 may then monitor signals generated by both its microphone and vision sensor 370. Signals generated by the microphone may be analyzed, e.g., by scoring engine 116, to make sure subject 101 is sufficiently engaged in the working memory task of repeatedly reciting the pledge. Meanwhile, signals generated by vision sensor 370 may be analyzed to determine if and when subject 101 holds up three fingers (within a field of view of vision sensor). If subject 101 completes the prospective memory task in a timely manner (i.e., holds up three fingers at or close to the assigned point in time), his or her measure of prospective memory may be increased (e.g., incremented as described previously). However, if he or she fails to hold up three fingers in a timely manner, or holds up the wrong number of fingers, or even stops reciting the pledge for too long a period, his or her measure of prospective memory may be decreased (or at least not increased).

In FIG. 3B, working memory task instructions are provided to subject 101, “Please look at the screen and identify differences between the two depicted scenes. In the meantime, please clap your hands twice two minutes after a yellow star appears on the screen.” After subject 101 affirms, two or more digital images (not depicted, e.g., showing similar scenes with subtle differences) may be rendered on another client device 306B, which in this case takes the form of a smart television or regular television equipped with a smart “dongle.” While subject 101 vocally identifies differences they perceive between the multiple displayed images (the subject's utterances may be detected by the microphone of either client device), subject 101 may also watch to await the appearance of a yellow star (not depicted). When the star appears on client device 306B, subject 101 must then wait two minutes (which they can monitor using clock 354) until they clap their hands twice. The clapping may be detected by the microphone of client device 306 (or by a microphone of another client device, such as client device 306B). Again, the actual time the clapping was detected may be compared to the assigned time. Any difference (or delta) between the two may be used to compute a measure of prospective memory of subject 101.

Although examples described herein (including in association with the above-described algorithm) have used numeric points in the form of integers to calculate a measure of prospective memory, this is not meant to be limiting. In some implementations, linear weighting of the delta between actual and assigned points in time may be employed. For example, if the delta falls within a first range relatively close to the assigned time (e.g., within ten seconds), the subject may be awarded a whole point. If the delta falls outside of the first range but within a second range, a half point may be awarded. And so on.

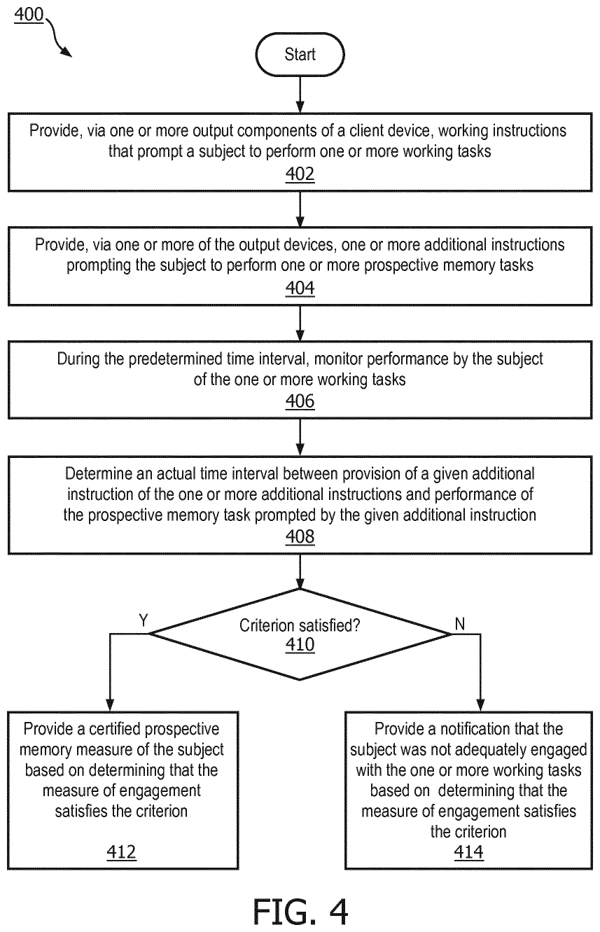

FIG. 4 illustrates a flowchart of an example method 400 for practicing selected aspects of the present disclosure, in accordance with various embodiments. For convenience, the operations of FIG. 4 will be described as being performed by a system. Other implementations may include additional steps than those illustrated in FIG. 4, may perform step(s) of FIG. 4 in a different order and/or in parallel, and/or may omit one or more of the steps of FIG. 4.

At block 402, the system may provide, e.g., via one or more output components of a client device (e.g., 106, 206, 306) such as a speaker or touchscreen display, working memory instructions that prompt a subject to perform one or more working memory tasks. In various implementations, the one or more working tasks may be selected to occupy the working memory of the subject.

At block 404, the system may provide, e.g., via one or more of the output devices, one or more additional instructions prompting the subject to perform one or more prospective memory tasks. Each respective additional instruction of the one or more additional instructions may prompt the subject to perform a respective one of the prospective memory tasks a predetermined time interval after provision of the respective additional instruction. For example the subject may be instructed to perform some task in x minutes, or y seconds after some sort of cue (which can be output using any output modality).

At block 406, the system may, e.g., during the predetermined time interval, monitor performance by the subject of the one or more working memory tasks. In various embodiments, the monitoring may include determining a measure of engagement between the subject and one or more input devices operated by the subject to perform the one or more working memory tasks. In the scenario of FIG. 2, for instance, interaction with touchscreen 250 may be monitored, e.g., to ensure the subject touches a graphical element more than some minimum frequency. In FIGS. 3A and 3B, audible input provided by the subject and captured using a microphone may be monitored. In either case, a frequency, duration, and/or any other measure of the subject's engagement with the input component(s) may be monitored and/or compared to various thresholds.

At block 408, the system may determine an actual time interval between provision of a given additional instruction of the one or more additional instructions and performance of the prospective memory task prompted by the given additional instruction. In some embodiments, the system may set a timer when the instruction is provided and measure the time based on the timer. In other embodiments, timestamps associated with the instruction and performance of the task may be detected.

At block 410, the system may determine whether the measure of engagement (determined at block 406) satisfies a criterion (e.g., a minimum frequency threshold). If the answer is yes, then at block 412, the system may (conditionally based on the outcome of 410) provide a certified prospective memory measure of the subject based on determining that the measure of engagement satisfies the criterion. In various embodiments, the certified prospective memory measure may be calculated based at least in part on the actual time interval.

If the answer at block 410 is no, on the other hand, then at block 414, the system may (again, conditionally on an outcome of block 410) provide a notification that the subject was not adequately engaged with the one or more working memory tasks based on determining that the measure of engagement satisfies the criterion. In other embodiments, in addition to or instead of providing a notification, the subject's performance of the prospective memory task may be discarded and/or discounted.

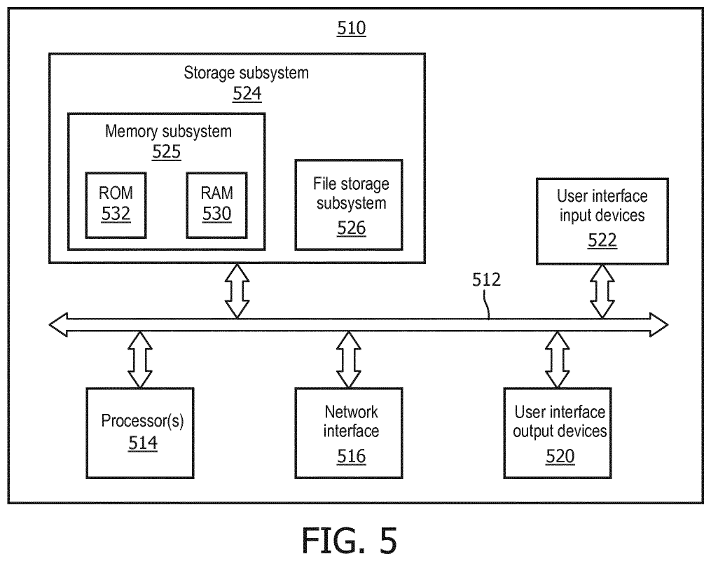

FIG. 5 is a block diagram of an example computing device 510 that may optionally be utilized to perform one or more aspects of techniques described herein. Computing device 510 typically includes at least one processor 514 which communicates with a number of peripheral devices via bus subsystem 512. These peripheral devices may include a storage subsystem 524, including, for example, a memory subsystem 525 and a file storage subsystem 526, user interface output devices 520, user interface input devices 522, and a network interface subsystem 516. The input and output devices allow user interaction with computing device 510. Network interface subsystem 516 provides an interface to outside networks and is coupled to corresponding interface devices in other computing devices.

User interface input devices 522 may include a keyboard, pointing devices such as a mouse, trackball, touchpad, or graphics tablet, a scanner, a touchscreen incorporated into the display, audio input devices such as voice recognition systems, microphones, and/or other types of input devices. In general, use of the term “input device” is intended to include all possible types of devices and ways to input information into computing device 510 or onto a communication network.

User interface output devices 520 may include a display subsystem, a printer, a fax machine, or non-visual displays such as audio output devices. The display subsystem may include a cathode ray tube (CRT), a flat-panel device such as a liquid crystal display (LCD), a projection device, or some other mechanism for creating a visible image. The display subsystem may also provide non-visual display such as via audio output devices. In general, use of the term “output device” is intended to include all possible types of devices and ways to output information from computing device 510 to the user or to another machine or computing device.

Storage subsystem 524 stores programming and data constructs that provide the functionality of some or all of the modules described herein. For example, the storage subsystem 524 may include the logic to perform selected aspects of the method of FIG. 4, as well as to implement various components depicted in FIG. 1.

These software modules are generally executed by processor 514 alone or in combination with other processors. Memory 525 used in the storage subsystem 524 can include a number of memories including a main random access memory (RAM) 530 for storage of instructions and data during program execution and a read only memory (ROM) 532 in which fixed instructions are stored. A file storage subsystem 526 can provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem 526 in the storage subsystem 524, or in other machines accessible by the processor(s) 514.

Bus subsystem 512 provides a mechanism for letting the various components and subsystems of computing device 510 communicate with each other as intended. Although bus subsystem 512 is shown schematically as a single bus, alternative implementations of the bus subsystem may use multiple busses.

Computing device 510 can be of varying types including a workstation, server, computing cluster, blade server, server farm, or any other data processing system or computing device. Due to the ever-changing nature of computers and networks, the description of computing device 510 depicted in FIG. 5 is intended only as a specific example for purposes of illustrating some implementations. Many other configurations of computing device 510 are possible having more or fewer components than the computing device depicted in FIG. 5.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be understood that certain expressions and reference signs used in the claims pursuant to Rule 6.2(b) of the Patent Cooperation Treaty (“PCT”) do not limit the scope.

1. A method for conducting a prospective memory test on a subject, the method implemented using one or more processors and comprising:
providing, via one or more output devices, working memory instructions that prompt the subject to perform one or more working memory tasks, wherein the one or more working memory tasks are selected to occupy the working memory of the subject;
providing, via one or more of the output devices, one or more additional instructions prompting the subject to perform one or more prospective memory tasks, wherein each respective additional instruction of the one or more additional instructions prompts the subject to perform a respective one of the prospective memory tasks a predetermined time interval after provision of the respective additional instruction;
during the predetermined time interval, monitoring performance by the subject of the one or more working memory tasks, wherein the monitoring includes determining a measure of engagement between the subject and one or more input devices operated by the subject to perform the one or more working memory tasks;
determining an actual time interval between provision of a given additional instruction of the one or more additional instructions and performance of the prospective memory task prompted by the given additional instruction;
determining whether the measure of engagement satisfies a criterion;
conditionally providing a certified prospective memory measure of the subject based on determining that the measure of engagement satisfies the criterion, wherein the certified prospective memory measure is calculated based at least in part on the actual time interval; and
conditionally providing a notification that the subject was not adequately engaged with the one or more working memory tasks based on determining that the measure of engagement satisfies the criterion.

2. The method of claim 1, further comprising rendering, on a display, a graphical user interface, wherein the graphical user interface is operable by the subject to participate in the prospective memory test.

3. The method of claim 2, wherein providing the working memory instructions includes providing, as part of the graphical user interface, one or more working interactive elements that are operable by the subject to perform the one or more working memory tasks.

4. The method of claim 2, wherein providing the one or more additional instructions includes providing, as part of the graphical user interface, one or more prospective memory interactive elements that are operable by the subject to perform the one or more prospective memory tasks.

5. The method of claim 4, wherein the actual time interval occurs between provision of the one or more prospective memory interactive elements and operation of the one or more prospective memory interactive elements by the subject.

6. The method of claim 2, further comprising rendering, as part of the graphical user interface, a clock.

7. The method of claim 1, wherein the respective additional instruction is provided immediately prior to the one or more working memory instructions.

8. The method of claim 1, wherein the respective additional instruction is provided after the one or more working memory instructions.

9. A system for facilitating a prospective memory test, the system comprising one or more processors and memory storing instructions that, in response to execution of the instructions by the one or more processors, cause the one or more processors to perform the following operations:
providing, via one or more output devices, working memory instructions that prompt a subject to perform one or more working memory tasks, wherein the one or more working memory tasks are selected to occupy the working memory of the subject;
providing, via one or more of the output devices, one or more additional instructions prompting the subject to perform one or more prospective memory tasks, wherein each respective additional instruction of the one or more additional instructions prompts the subject to perform a respective one of the prospective memory tasks a predetermined time interval after provision of the respective additional instruction;
during the predetermined time interval, monitoring performance by the subject of the one or more working memory tasks, wherein the monitoring includes determining a measure of engagement between the subject and one or more input devices operated by the subject to perform the one or more working memory tasks;
determining an actual time interval between provision of a given additional instruction of the one or more additional instructions and performance of the prospective memory task prompted by the given additional instruction;
determining whether the measure of engagement satisfies a criterion; conditionally providing a certified prospective memory measure of the subject based on determining that the measure of engagement satisfies the criterion, wherein the certified prospective memory measure is calculated based at least in part on the actual time interval; and
conditionally providing a notification that the subject was not adequately engaged with the one or more working memory tasks based on determining that the measure of engagement satisfies the criterion.

10. The system of claim 9, further comprising rendering, on a display, a graphical user interface, wherein the graphical user interface is operable by the subject to participate in the prospective memory test.

11. The system of claim 10, wherein providing the working memory instructions includes providing, as part of the graphical user interface, one or more working interactive elements that are operable by the subject to perform the one or more working memory tasks.

12. The system of claim 11, wherein providing the one or more additional instructions includes providing, as part of the graphical user interface, one or more prospective memory interactive elements that are operable by the subject to perform the one or more prospective memory tasks.

13. The system of claim 12, wherein the actual time interval occurs between provision of the one or more prospective memory interactive elements and operation of the one or more prospective memory interactive elements by the subject.

14. The system of claim 10, further comprising rendering, as part of the graphical user interface, a clock.

15. The system of claim 9, wherein the respective additional instruction is provided immediately prior to the one or more working instructions.
